(19) United States
(12) Patent Application Publication
(10) Pub. No.: US 2002/0197229 A1
(43) Pub. Date: Dec. 26, 2002

(54) Heat-crosslinkable cosmetic composition

(75) Inventors: Pascale Bernard, Sucy-en-Brie (FR); Roland Ramin, Paris (FR); Jean Mondet, Aulnay-sous-Bois (FR)

Correspondence Address: STEPTOE & JOHNSON LLP, 1330 Connecticut Ave., N.W., Washington, DC, 20036, US

(73) Assignee: L'OREAL, Paris (FR)

(21) Appl. No.: 10/115,280

(22) Filed: Apr. 4, 2002

(30) Foreign Application Priority Data
Apr. 6, 2001 (FR) 01 04681

(51) Int. Cl. A61K 7/06; A61K 7/11; C08G 18/81

(52) U.S. Cl. 424/70.17; 528/45

(57) ABSTRACT

The invention relates to a heat-crosslinkable cosmetic composition comprising, in a cosmetically acceptable medium, (a) at least one compound comprising at least two functions containing labile hydrogen, and (b) at least one compound comprising at least two blocked isocyanate functions, which can be unblocked by heating, the average functionality of the system, that is to say the total number of functions containing labile hydrogen and of blocked isocyanate functions relative to the total number of molecules of compounds (a) and (b), being strictly greater than 2, and also to a process for coating keratin substrates using such a composition.

The present invention relates to heat-crosslinkable cosmetic compositions, in particular heat-crosslinkable nail varnishes, and also to a process for coating keratin materials using these compositions.

In the field of nail varnishes, the film deposited on the nails is generally obtained by simple drying—involving no chemical reaction—of cosmetic compositions mainly containing film-forming polymers and volatile organic solvents, and optionally pigments or colorants.

These deposits do not always have a satisfactory staying power and have the drawback of needing to be freshened at regular intervals, which places an unpleasant constraint on the user.

The Applicant set itself the objective of developing cosmetic compositions, in particular nail varnishes, capable of being crosslinked in situ and thus of forming films with better chemical resistance and mechanical strength than those of the prior art.

In this perspective, the Applicant did not envisage using two reagents kept in separate containers and mixed together immediately before application, since such a device is very impractical.

The storage, in the same container, of two reagents capable of reacting together at a precise moment thus posed the problem of the premature crosslinking of the system, which had to be avoided at all costs.

The Applicant has solved this problem by reversibly inactivating, with a blocking agent, one of the two types of reactive function involved in the crosslinking reaction. This blocking agent is removed, after applying the cosmetic composition to the substrate, by the action of heat and the reactive functions thus freed can then react with the “coreactive” functions also present in the composition.

Consequently, one subject of the present invention is a heat-crosslinkable cosmetic composition comprising, in a cosmetically acceptable medium, at least one first compound (a) comprising at least two functions containing labile hydrogen, and at least one second compound (b) comprising at least two blocked isocyanate functions, which can be unblocked by heating, the average functionality of the system, that is to say the total number of functions containing labile hydrogen and of blocked isocyanate functions relative to the total number of molecules of compounds (a) and (b), being strictly greater than 2.

A subject of the invention is also a process for coating a keratin substrate, comprising the heating of the cosmetic film applied at a temperature that is sufficient to bring about unblocking of some or all of the blocked isocyanate functions borne by compound (b), so as to allow the deposit to crosslink.

These heat-crosslinkable cosmetic compositions combine good stability over time at room temperature and excellent reactivity after heating, allowing rapid hardening of the films applied.

As indicated above, the heat-crosslinkable cosmetic compositions contain two types of compound:

a first compound, referred to as compound (a), comprising at least two functions containing labile hydrogen, and

a second compound, referred to as compound (b), comprising at least two isocyanate functions inactivated by the presence of a blocking agent and capable of being unblocked, that is to say activated, by the effect of heat.

In order for this reactive system formed by compounds (a) and (b) to be able to form a crosslinked macromolecular network, its average functionality, that is to say the total number of functions containing labile hydrogen and of blocked isocyanate functions relative to the total number of molecules of compounds (a) and (b), must be greater than 2. Specifically, an average functionality or less than or equal to 2 would simply give a linear or branched polymer system.

In order to obtain a satisfactory crosslinking effect, the average functionality of the crosslinking system for the cosmetic compositions of the present invention is preferably at least equal to 2.2 and better still between 2.5 and 100.

In one embodiment of the cosmetic compositions of the present invention, a more or less large fraction or all of the compounds (a) and/or (b) may respectively bear, in addition to the functions containing labile hydrogen and/or blocked isocyanate functions intrinsic thereto, one or more “coreactive” functions. In other words, some or all of the compounds (a) can bear, in addition to the functions containing labile hydrogen, a certain number of isocyanate functions inactivated with a blocking agent and, similarly, some or all of the compounds (b) can bear, in addition to the blocked isocyanate functions, a certain number of functions containing labile hydrogen.

This means that the present invention also encompasses, in one specific embodiment, compositions in which all of the compounds forming the crosslinking system comprise both functions containing labile hydrogen and blocked isocyanate functions.

According to the present invention, the functions containing labile hydrogen are preferably chosen from primary amine (—NH₂), secondary amine (>NH), hydroxyl (—OH), carboxylic acid (—COOH) and thiol (—SH) functions.

Among these functions, the primary and secondary amine functions and the hydroxyl functions are most particularly preferred.

The blocked isocyanate functions capable of reacting, after heat activation, with the functions containing labile hydrogen, preferably correspond to the formula

—NH—C(=O)—B

in which B represents a radical derived from a blocking agent BH chosen from organic compounds comprising one or more, and preferably only one labile hydrogen atom.

The blocking agents must be capable of preventing the subsequent reaction of the isocyanate groups at room temperature, or more generally at a temperature below 45° C., with any other molecule containing labile hydrogen atoms, but must allow this reaction at a higher temperature, that is to say generally greater than or equal to 50° C., after thermal unblocking of the isocyanate function.

Examples of suitable blocking agents that may be mentioned include

a) alcohols, especially monoalcohols, containing from 1 to 10 carbon atoms, preferably tertiary monoalcohols containing from 4 to 10 carbon atoms and in particular from 4 to 6 carbon atoms,

b) phenols, especially monophenols, especially containing from 6 to 50 carbon atoms and preferably from 6 to 20 carbon atoms, such as ortho-cresol, para-cresol, 2,6-dimethylphenol, 2-tert-butylphenol, 2-[(dimethylamino)methyl]-phenol and methyl salicylate,

c) amides and in particular cyclic amides, especially containing from 3 to 50 carbon atoms, such as caprolactam, methylacetamide, imides such as succinimide and succinimides substituted especially with one or two C₁₋₆ alkyl radicals,

d) oximes, especially C₂₋₅ oximes, such as acetone-oxime, methyl isopropyl ketone-oxime, methyl isobutyl ketone-oxime, diisobutyl ketone-oxime and diisopropyl ketone-oxime,

e) β-dicarbonyl compounds, especially β-diesters, β-diketones and β-keto esters, such as C₁₋₆ dialkyl malonates, for example diethyl malonate, C₁₋₆ alkyl acetoacetates, for example ethyl acetoacetate or tert-butyl acetoacetate, or 2,4-pentanedione,

f) pyrazoles, in particular 3-methylpyrazole and 3,5-dimethylpyrazole,

g) esters of hydroxamic acid and of C₁₋₆ alcohols,

h) triazoles such as benzotriazole,

i) imidazolines such as 2-phenylimidazoline, 2,4-dimethylimidazoline and 4-methylimidazoline,

j) tetrahydropyrimidines, and

k) imidazoles such as 2-ethyl-4-methylimidazole.

These blocking reagents and the suitable reaction conditions are described, for example, in the articles: “Blocked Isocyanates” by Zeno W. in Progress in Organic Coatings, 3, pages 73-99 (1975) and “New Developments in the Field of Blocked Isocyanates” Zeno W., in Progress in Organic Coatings, 9, pages 3-28 (1981).

The reactions for inactivating and activating the isocyanate functions borne by the compound(s) (a), that is to say the reaction between the free isocyanate functions and the blocking agent BH, form a reaction equilibrium. To prevent the reactive isocyanate functions, released by heating, from reacting again with the labile hydrogen of the blocking agent, that is to say to shift the reaction equilibrium towards the activation of the isocyanate functions, it is desirable for the blocking agent to be removed from the film of cosmetic composition deposited and heated. This removal may take place, for example, by evaporation of the blocking agent.

In one preferred embodiment of the invention, the blocking agent BH consequently has a boiling point of greater than 45° C. and less than or equal to 100° C. and in particular between 45° C. and 80° C.

The compounds (b) bearing at least two blocked isocyanate functions are obtained by reaction between a suitable blocking agent BH and a compound comprising at least two free isocyanate functions. These compounds comprising at least two free isocyanate functions are known in the art. They may be diisocyanates or polyisocyanates of low molecular mass, or else synthetic oligomers or polymers of any chemical nature, obtained by polyaddition, polycondensation and/or grafting, or polymers of natural origin, optionally chemically modified, bearing two or more isocyanate functions either at the chain ends or on the side groups.

Non-limiting examples of such compounds that may be mentioned include:

a) aliphatic, cycloaliphatic and/or aromatic diisocyanates especially containing from 4 to 100 and preferably from 4 to 30 carbon atoms, such as hexamethylene diisocyanate, isophorone diisocyanate, toluene diisocyanate and diphenylmethane diisocyanate,

b) aliphatic, cycloaliphatic and/or aromatic triisocyanates especially containing from 4 to 50 and preferably from 4 to 30 carbon atoms, such as

the triisocyanates obtained by reaction between a triol and an excess of diisocyanate, especially those of formula:

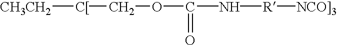

the isocyanatobiurets of formula:

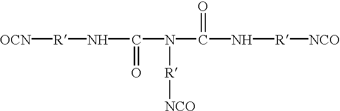

the isocyanurates of formula:

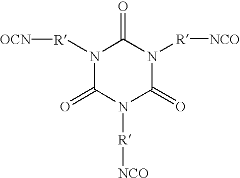

in which each R′ independently represents a linear, branched or cyclic hydrocarbon-based radical containing from 2 to 30 carbon atoms. Such non-blocked triisocyanates are sold, for example, under the name Desmodur® L and Desmodur® N by the company Bayer and under the name Tolonate® HDB-LV by the company Rhodia.

c) polycondensates containing terminal or lateral isocyanate groups, such as polyurethanes, polyureas, polyethers, polyesters, polyamides and perfluoropolyethers. Such polycondensates containing isocyanate groups are especially described in documents U.S. Pat. No. 5,281,654, U.S. Pat. No. 6,106,578, U.S. Pat. No. 6,100,310, WO 99/48942, CN-A-1 093 377, JP-A-04-077581 and FR-A-1 573 596.

d) polymers resulting from the copolymerization of vinyl, allylic and/or (meth)acrylic monomers and of ethylenically unsaturated comonomers comprising a free isocyanate function, such as isocyanatoethyl methacrylate.

e) silicones containing isocyanate groups, especially amino silicones comprising isocyanate groups, such as those described in EP-A-814 764.

Compounds containing blocked isocyanate groups are sold under the names Vestanat® B1358A, Vestanat® B1370, Vestanat® B1358/100 by the company Creanova, under the names Tolonate® D2 or D2R565 by the company Rhodia, under the name Desmodur® Z4470 by the company Bayer and under the names Trixene® B1 7951 and Trixene® B1 7982 by the company Baxenden.

It is also possible to use, as compounds (b), compounds containing “self-blocked” isocyanate functions, such as urethane-diones, obtained by dimerizing 2 molecules of diisocyanates, or alternatively tris((C₁₋₆ alkoxy)-carbonylamino)triazines such as the product of condensation of melanine, of dimethyl carbonate and of butanol. Such compounds are especially sold under the name Cylink® 2000 by the company Cyteck.

The compounds (a) bearing at least two functions containing labile hydrogen used in the present invention are also known. These may be organic compounds of low molecular mass or synthetic oligomers or polymers, obtained by polyaddition, polycondensation and/or grafting, or chemically modified natural polymers.

Families of compounds that may be mentioned include diols and polyols, primary and/or secondary diamines and polyamines, amino alcohols and polymers comprising at least two functions containing labile hydrogen.

Specific examples of compounds (a) are: C₁₋₄ alkylene glycols, glycerol, trimethylolpropane, pentaerythritol, poly(C₁₋₄ alkylene) glycols such as polyethylene glycol or polypropylene glycol or copolymers thereof, the product of condensation of propylene glycol and of trimethylolpropane, castor oil, phytanetriol, sugars and carbohydrates such as sucrose or cellulose, ethylenediamine, 1,3-diaminopropane, lysine, 2-amino-2-methyl-1-propanol, poly(alkylenoxy)diamines, such as Jeffamine® products sold by the company Texaco, nitrocellulose, cellulose esters, especially those with a degree of substitution of less than 3, such as cellulose acetobutyrate and cellulose acetopropionate, cellulose ethers such as hydroxyethylcellulose, carboxymethylcellulose, hydroxypropylcellulose or ethylcellulose, polyester resins, silicones, perfluoropolyethers, alkyds and polyketones with hydroxylated end groups, poly(vinyl alcohol) and copolymers based on vinyl alcohol, copolymers of allyl alcohol, copolymers based on C₂₋₁₀ hydroxyalkyl (meth)acrylate, for instance 2-hydroxyethyl (meth)acrylate or 2-hydroxypropyl (meth)acrylate, sold especially under the name Joncryl® SCX 910 by the company Johnson Polymer or under the name Crodoplast® AC 5725 by the company Croda, copolymers based on vinylamine or allylamine, silicones and perfluoroethers with primary or secondary amine end groups, hyperbranched dendrimers or polymers with hydroxyl or primary amine end groups, such as the hyperbranched polyesters with hydroxyl end groups sold by the company Perstorp under the names Boltorn® H40 TMP Core and HBP Polyol® 3G (described in international patent applications WO 93/17060 and WO 96/12754), or alternatively dendrimers of polyamidoamine type with primary amine end groups, described in the article by Tomalia, Angewandte Chemie, Int. Engl. Ed., Vol. 29, No. 2, pages 138-175.

The concentration of compounds (a) and (b) in the cosmetic compositions of the present invention is preferably between 1% and 50% by weight and more preferably between 2% and 40% by weight.

Moreover, the compounds (a) and compounds (b) are present in amounts such that the ratio of the number of functions containing labile hydrogen to the number of blocked isocyanate functions is greater than or equal to 1 and especially between 1 and 1.5.

The chemical crosslinking system containing compounds (a) and (b) described above is stable at room temperature and may be activated by increasing the temperature. The temperature required to activate the blocked isocyanate functions depends, of course, on the chemical nature of the blocking agent and on that of the di- or polyisocyanate. When the minimum activation temperature of a certain type of blocked function proves to be too high, that is to say incompatible with biological substrates, it is possible to lower this activation temperature by adding suitable catalysts.

The range of temperatures used for hardening the films preferably ranges from 45° C. to 150° C. and in particular from 50° C. to 100° C.

The heat-crosslinkable cosmetic compositions of the present invention thus preferably contain one or more catalysts capable of accelerating the thermal unblocking reaction of the blocked isocyanate functions. These catalysts are especially chosen from tertiary amines such as diazabicyclo[2.2.2] octane, quinuclidine and 3,3,6,9,9-pentamethyl-2,10-diazabicyclo[4.4.0] dec-1-ene, tin chloride, organometallic compounds such as metallic acetonylacetates, organometallic tin compounds, calcium hexanoate, calcium 2-ethylhexanoate, calcium octanoate and calcium linoleate, dibutyltin dilaurate, bismuth tris(2-ethylhexanoate) and zinc bis(2-ethylhexanoate). These catalysts and conditions for using them are also described in the article “Blocked Isocyanates” by Zeno W. in Progress in Organic Coatings, 3, pages 73-99 (1975).

According to the present invention, the catalyst concentration is preferably between 0.1% and 5% by weight and more particularly between 0.2% and 3% by weight relative to the total weight of compound (b) bearing blocked isocyanate groups.

The heat-crosslinkable cosmetic compositions of the present invention may contain one or more solvents, that are preferably volatile, chosen from water and physiologically acceptable organic solvents, among which mention may be made of

ketones that are liquid at room temperature, such as methyl ethyl ketone, methyl isobutyl ketone, diisobutyl ketone, isophorone, cyclohexanone and acetone,

alcohols that are liquid at room temperature, such as ethanol, isopropanol, diacetone alcohol, 2-butoxy-ethanol or cyclohexanol,

glycols that are liquid at room temperature, such as ethylene glycol, propylene glycol, pentylene glycol and glycerol,

propylene glycol ethers that are liquid at room temperature, such as propylene glycol monomethyl ether, propylene glycol monomethyl ether acetate and dipropylene glycol mono-n-butyl ether,

short-chain esters (containing in total from 3 to 8 carbon atoms), such as ethyl acetate, methyl acetate, propyl acetate, n-butyl acetate and isopentyl acetate,

alkanes that are liquid at room temperature, such as decane, heptane, dodecane and cyclohexane,

aromatic hydrocarbons that are liquid at room temperature, such as toluene and xylene,

silicones that are liquid at room temperature, and

mixtures thereof.

The solvent content in the composition may range from 0.1% to 80% by weight relative to the total weight of the composition and preferably from 1% to 60% by weight.

According to one particular embodiment of the composition according to the invention, the composition is free of solvent.

The photo-crosslinkable cosmetic compositions of the present invention may also contain adjuvants and additives commonly used in nail varnishes, chosen especially from pigments and colorants, plasticizers, coalescers, preserving agents, waxes, thickeners, fragrances, UV screening agents, nail care cosmetic active agents, spreading agents, antifoams, surfactants and dispersants.

Needless to say a person skilled in the art will take care to select these optional adjuvants and additives such that the advantageous properties of the compositions according to the invention are not, or are virtually not, adversely affected by the envisaged addition.

A subject of the invention is also a process for coating keratin substrates. This process generally comprises the steps consisting

in applying to the keratin substrate a coat of a heat-crosslinkable cosmetic composition described above,

in optionally leaving the deposited cosmetic composition to dry, and

in subjecting the deposited cosmetic composition, optionally dried, to heating up to a temperature that is sufficient and for a time that is sufficient to bring about unblocking of some or all of the blocked isocyanate functions borne by compound (a), so as to allow the partial or total crosslinking of the deposit.

The keratin substrates that may receive heat-crosslinkable coatings according to the present invention are, in particular, the nails, the hair, the eyelashes and the eyebrows, but makeup accessories such as false eyelashes, false nails or wigs may also be coated.

The degree of crosslinking of the cosmetic coatings according to the present invention does not depend only on the temperature, but, of course, also on the duration of heating. The longer this heating, the lower the minimum temperature required for crosslinking.

The heating temperature, that is to say the local temperature obtained in situ in the coat of applied cosmetic composition, is preferably between 45° C. and 150° C. and in particular between 50° C. and 100° C., this temperature preferably being maintained for about 2 minutes to 1 hour and in particular between 5 and 15 minutes.

The heating of the coat of cosmetic composition may take place using any suitable source of heat. Examples of such a source of heat that may be mentioned include a heating chamber, a device for projecting heat such as a hairdryer, or a source of radiation allowing the temperature of the composition to be raised, such as an infrared lamp.

The use of an infrared lamp represents a preferred embodiment of the invention, since it makes it possible to obtain high local temperatures on the coat of varnish applied, without this involving an excessive heating of the cosmetic support, that is to say of the nail or the hair. The use of an infrared lamp also makes it possible to obtain a differential crosslinking of the applied coat, that is to say a deposit that is more crosslinked at the surface than deep down, which gives coatings with good mechanical strength, good adhesion to the support and that are easily removed with standard dissolvers.

The invention is described in greater detail by means of the examples below.

### EXAMPLES

### Example 1

A nail varnish composition is prepared from the following ingredients:

| | | |
|---|---|---|
| Joncryl ® SCX 910 (Johnson Polymer) | 21 | g |
| (acrylic polymer containing hydroxyl groups) | | |
| Tolonate ® D2 (Rhodia) | 9 | g |
| (polyisocyanate blocked with methyl ethyl | | |
| ketone) | | |
| Aerosil ® R972 (Degussa) | 0.5 | g |
| (fumed silica) | | |
| Byk ® 162 (Byk) | 1 | g |
| (surfactant) | | |
| Modaflow ® (Monsanto) | 0.1 | g |
| (surfactant) | | |
| Pigments | 3 | g |
| Butyl acetate | 20 | g |
| Ethyl acetate | qs 100 | g |

A film of this composition is applied to the nails and is left to dry for about 5 minutes so as to allow the solvent to evaporate off. The temperature of the film is then raised to 65° C. for 3 minutes using a hairdryer.

The film obtained is touch-dry, non-sticky, glossy and has good properties of adhesion to the substrate and good staying power over time.

### Example 2

A nail varnish composition is prepared from the following ingredients:

| | | |
|---|---|---|
| Crodoplast ® AC 5725 (Croda) | 16.4 | g |
| (acrylic polymer containing hydroxyl groups) | | |
| Trixene ® B1 7951 (Baxenden) | 7.1 | g |
| (isophorone diisocyanate blocked with | | |
| 3,5-dimethylpyrazole) | | |
| Trixene ® B1 7982 (Baxenden) | 6.5 | g |
| (hexamethylene dilsocyanate blocked with | | |
| 3,5-dimethylpyrazole) | | |
| Dibutyltin dilaurate | 0.01 | g |
| Aerosil ® R972 (Degussa) | 0.5 | g |
| (fumed silica) | | |
| Pigments | 3 | g |
| Modaflow ® (Monsanto) | 0.1 | g |
| (surfactant) | | |
| Heptane | 25 | g |
| Ethyl acetate | qs 100 | g |

A film of this composition is applied to the nails and is left to dry for about 5 minutes so as to allow the solvent to evaporate off. The temperature of the surface of the film is then raised using an infrared radiation which gives regular pulses of a temperature of at least 80° C. for a few seconds to initiate the unblocking of the isocyanate functions. The film obtained has properties identical to those of the film obtained in Example 1.

1. Heat-crosslinkable cosmetic composition comprising, in a cosmetically acceptable medium,
(a) at least one compound comprising at least two functions containing labile hydrogen, and
(b) at least one compound comprising at least two blocked isocyanate functions, which can be unblocked by heating,
the average functionality of the system, that is to say the total number of functions containing labile hydrogen and of blocked isocyanate functions relative to the total number of molecules of compounds (a) and (b), being strictly greater than 2.

2. Heat-crosslinkable cosmetic composition according to claim 1, characterized in that some or all of the compounds (a) also bear one or more blocked isocyanate functions and/or in that some or all of the compounds (b) also bear one or more functions containing labile hydrogen.

3. Heat-crosslinkable cosmetic composition according to claim 1 or 2, characterized in that the functions containing labile hydrogen, borne by compound (a), are chosen from primary amine (—NH₂), secondary amine (>NH), hydroxyl (—OH), carboxylic acid (—COOH) and thiol (—SH) functions.

4. Heat-crosslinkable cosmetic composition according to claims 1 to 3, characterized in that the blocked isocyanate functions of compound (b) correspond to the formula
—NH—C(=O)—B
in which B represents a radical derived from a blocking agent BH chosen from organic compounds comprising one or more, and preferably only one labile hydrogen atom.

5. Heat-crosslinkable cosmetic composition according to claim 4, characterized in that the blocking agent BH is chosen from monoalcohols, monophenols, amides, oximes, β-dicarbonyl compounds, pyrazoles, esters of hydroxyamic acid and of C₁₋₆ alcohols, triazoles, imidazolines, tetrahydropyrimidines and imidazoles.

6. Heat-crosslinkable cosmetic composition according to claim 4 or 5, characterized in that the blocking agent BH has a boiling point of greater than 45° C. and less than or equal to 100° C., preferably between 45° C. and 80° C.

7. Heat-crosslinkable cosmetic composition according to any one of the preceding claims, characterized in that compound (b) bearing at least two blocked isocyanate functions is obtained by reaction between a blocking agent BH defined in claims 4 to 6 and a compound comprising at least two isocyanate functions chosen from
a) aliphatic, cycloaliphatic and/or aromatic diisocyanates especially containing from 4 to 50 and preferably from 4 to 30 carbon atoms, such as hexamethylene diisocyanate, isophorone diisocyanate, toluene diisocyanate and diphenylmethane diisocyanate,
b) aliphatic, cycloaliphatic and/or aromatic triisocyanates especially containing from 4 to 100 and preferably from 4 to 30 carbon atoms, such as those of formula 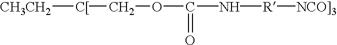
in which each R′ independently represents a linear, hollow branched or cyclic hydrocarbon-based radical containing from 2 to 30 carbon atoms,
c) polycondensates containing terminal or lateral isocyanate groups, such as polyurethanes, polyureas, polyethers, polyesters, polyamides and perfluoropolyethers,
d) polymers resulting from the copolymerization of vinyl, allylic and/or (meth)acrylic monomers and of ethylenically unsaturated comonomers comprising a free isocyanate function,
e) silicones containing isocyanate groups.

8. Heat-crosslinkable cosmetic composition according to any one of the preceding claims, characterized in that compound (a) is chosen from diols and polyols, primary and/or secondary diamines and polyamines, amino alcohols and polymers comprising at least two functions containing labile hydrogen.

9. Heat-crosslinkable cosmetic composition according to claim 8, characterized in that compound (a) is chosen from C₁₋₄ alkylene glycols, glycerol, trimethylolpropane, pentaerythritol, poly(C₁₋₄ alkylene) glycols such as polyethylene glycol or polypropylene glycol or copolymers thereof, the product of condensation of propylene glycol and of trimethylolpropane, castor oil, phytanetriol, sugars and carbohydrates such as sucrose or cellulose, ethylenediamine, 1,3-diaminopropane, lysine, 2-amino-2-methyl-l-propanol, poly(alkylenoxy)diamines, nitrocellulose, cellulose esters, cellulose ethers, polyester resins, silicones, perfluoropolyethers, alkyds and polyketones with hydroxylated end groups, poly(vinyl alcohol) and copolymers based on vinyl alcohol, copolymers of allyl alcohol, copolymers based on C₂₋₁₀ hydroxyalkyl (meth)acrylate, copolymers based on vinylamine or allylamine, silicones and perfluoroethers with primary or secondary amine end groups, hyperbranched dendrimers or polymers with hydroxyl or primary amine end groups.

10. Heat-crosslinkable cosmetic composition according to any one of the preceding claims, characterized in that compounds (a) and (b) represent from 1% to 50% by weight of the cosmetic composition.

11. Heat-crosslinkable cosmetic composition according to one of the preceding claims, characterized in that the total number of functions containing free hydrogen and of blocked isocyanate functions relative to the total number of molecules of compounds (a) and (b) is greater than 2.2 and preferably between 2.5 and 100.

12. Heat-crosslinkable cosmetic composition according to any one of the preceding claims, characterized in that it also comprises one or more compounds for catalysing the thermal unblocking reaction of the isocyanate functions of compound (b), chosen from tertiary amines such as diazabicyclo[2.2.2] octane, quinuclidine and 3,3,6,9,9-pentamethyl-2,10-diazabicyclo[4.4.0] dec-1-ene, tin chloride, organometallic compounds such as metallic acetonylacetates, organometallic tin compounds, calcium hexanoate, calcium 2-ethylhexanoate, calcium octanoate and calcium linoleate, dibutyltin dilaurate, bismuth tris(2-ethylhexanoate) and zinc bis(2-ethylhexanoate).

13. Heat-crosslinkable cosmetic composition according to claim 12, characterized in that the concentration of the compound for catalysing the thermal unblocking reaction of the isocyanate functions of compound (b) is between 0.1% and 5% by weight and preferably between 0.2% and 3% by weight relative to the total weight of compound (b) present.

14. Process for coating a keratin substrate, comprising the steps consisting
in applying to the keratin substrate a coat of a cosmetic composition according to one of the preceding claims,
in optionally leaving the deposited cosmetic composition to dry, and
in subjecting the deposited cosmetic composition, optionally dried, to heating up to a temperature that is sufficient and for a time that is sufficient to bring about unblocking of some or all of the blocked isocyanate functions borne by compound (a), so as to allow the crosslinking of the deposit.

15. Process according to claim 14, characterized in that the keratin substrate is the nail, the hair, the eyelashes and the eyebrows.

16. Process according to either of claims 14 and 15, characterized in that the cosmetic composition is heated to a temperature of between 45° C. and 150° C. and preferably between 50° C. and 100° C.

17. Process according to one of claims 14 to 16, characterized in that the duration of heating is between 2 minutes and 1 hour and preferably between 5 and 15 minutes.

18. Process according to one of claims 14 to 17, characterized in that the heating is carried out using a source of heat chosen from a heating chamber, a device for projecting heat such as a hairdryer, or a source of radiation allowing the temperature of the composition to be raised, such as an infrared lamp.
